(19) United States
(12) Patent Application Publication
(10) Pub. No.: US 2026/0194484 A1
(43) Pub. Date: Jul. 9, 2026

(54) GAS DETECTION SYSTEM WITH ENHANCED SELECTIVITY

(71) Applicant: INDUSTRY-ACADEMIC COOPERATION FOUNDATION, YONSEI UNIVERSITY, Seoul (KR)

(72) Inventors: Kyu Hyoung LEE, Seoul (KR); Min Young KIM, Seoul (KR); Chul Oh PARK, Seoul (KR); Ha Eun CHOA, Seoul (KR)

(21) Appl. No.: 19/437,248

(22) Filed: Dec. 30, 2025

(30) Foreign Application Priority Data
Jan. 6, 2025 (KR) 10-2025-0001530

(51) Int. Cl. G01N 27/12; G01N 33/00

(52) U.S. Cl. CPC G01N 27/125; G01N 33/0031

(57) ABSTRACT

The present disclosure relates to a gas sensing method with enhanced selectivity. Specifically, the present disclosure relates to a gas sensing method for more accurately distinguishing various types of gases through a process of analyzing a correlation of two or more kinetic parameters or static parameters from a resistance change of a gas sensor according to concentration under two or more concentration conditions, by changing a concentration of a captured external gas through dilution or concentration.

### CROSS-REFERENCE TO RELATED APPLICATION

This application claims priority to Korean Patent Application No. 10-2025-0001530 filed in the Korean Intellectual Property Office on Jan. 6, 2025, the disclosure of which is incorporated by reference herein in its entirety

### TECHNICAL FIELD

The present disclosure relates to a gas sensing method with improved selectivity for gases, and more particularly, to a gas sensing method for more accurately distinguishing various types of gases, the method including a process of analyzing a correlation of kinetic parameters or static parameters by using a change in resistance of a gas sensor according to concentration under two or more concentration conditions by varying a concentration of a gas.

### DISCUSSION OF RELATED ART

Gas sensors have been used in a wide range of fields, such as chemistry, pharmaceuticals, the environment, and medicine, and much more research is predicted to be conducted in the future. A technology for detecting harmful substances and pollutants in the air in real time is essential for maintaining a good living and working environment.

Currently, electrochemical gas sensors and semiconductor-type gas sensors are used to effectively detect low concentrations of harmful gases. In particular, semiconductor metal oxide-based gas sensor devices have advantages such as (i) low manufacturing cost, (ii) small size, (iii) relatively simple operating principle, (iv) variety of types, (v) excellent compatibility, and (vi) easy control of various compositions, shapes, and microstructures. Therefore, despite having a disadvantage where sensitivity and reliability are inferior to those of electrochemical-based gas sensors, they have been continuously researched with various dimensions, compositions, shapes, microstructures, and functionalizations.

Conventional gas sensors have a limitation in that it is difficult to clearly distinguish a type of gas because selectivity among various gas components is low. In general, when reactivity is high, a problem occurs where selectivity for various gas components decreases as reactivity for all gases increases.

Accordingly, the present inventors have developed a new gas sensing method to solve this problem of low selectivity of conventional gas sensors.

### SUMMARY

An object of the present disclosure is to provide a gas sensing method for more accurately distinguishing various types of gases, to solve the disadvantage of low selectivity of conventional gas sensors, through a process of analyzing the correlation between two or more kinetic parameters or static parameters using the resistance change of a gas sensor at different concentrations, which are varied by diluting or concentrating a captured external gas under two or more concentration conditions.

To achieve the above object, the present disclosure provides a gas sensing method including: i) capturing an external gas; ii) measuring a resistance value (Rₒ) of a gas sensor under a base gas condition to stabilize the gas sensor; iii) changing the concentration of the external gas and measuring resistance values (R_{g}) of the gas sensor under two or more concentration conditions with different concentrations; iv) calculating ratios (R_{g}/Rₒ) of the resistance value of the gas sensor under the base gas condition to the resistance values of the gas sensor under the two or more concentration conditions with different concentrations, and then deriving two or more kinetic parameters or static parameters from the ratios (R_{g}/Rₒ); and v) determining a type of the gas by comparing the correlation of the two or more kinetic parameters or static parameters calculated in the operation iv) with a standard correlation of two or more kinetic parameters or static parameters.

The gas sensing method of the present disclosure may easily and accurately distinguish the type of gas through a process of analyzing the correlation of two or more kinetic parameters or static parameters using the resistance change of the gas sensor according to concentration. Furthermore, the gas sensing method of the present disclosure may visualize the correlation of the kinetic parameters or the static parameters using a scatter plot. Therefore, the gas sensing method of the present disclosure may more easily and accurately distinguish the type of gas by representing a correlation of the kinetic parameters or the static parameters for a target external gas as a scatter plot and then comparing and analyzing it with a standard scatter plot for each gas.

Furthermore, the gas sensing method of the present disclosure may measure the resistance values of the gas sensor under more diverse conditions in which one or more of temperature, humidity, and light absorbance are additionally varied in addition to the concentration of the gas. Therefore, the gas sensing method of the present disclosure may more accurately distinguish various types of gases compared to a case where only the above-described concentration change is applied.

Furthermore, the gas sensing method of the present disclosure may more clearly distinguish various gases, thereby increasing gas selectivity, and provides more efficient detection performance compared to conventional gas sensors. In particular, the gas sensing method may have practical advantages in industrial sites and personal safety devices by enabling rapid and accurate identification of hazardous gases.

Furthermore, because the gas sensing method of the present disclosure is applicable to a multi-array system, a plurality of gas sensors may be arranged in parallel to simultaneously detect and analyze various types of gases. This multi-array application method complements the characteristics and selectivity of individual sensors, and by comprehensively processing data from a plurality of sensors, more accurate and precise gas identification is possible, and the stability and reliability of the entire system may be increased.

Furthermore, the gas sensing method of the present disclosure may be miniaturized, and through this, the system may be easily integrated into various everyday electronic devices such as smart watches, attachable sensors, and portable electronic devices. Thanks to these characteristics, the present disclosure provides a user-friendly and economical system and may have high commercial value in the field of gas detection and analysis.

### BRIEF DESCRIPTION OF THE DRAWINGS

FIG. 1 is a schematic diagram of a gas sensor system including a gas capturing unit, a gas dilution unit, a gas sensor unit, and a data analysis unit, according to an embodiment of the present disclosure.

FIG. 2 is a schematic diagram of a gas sensor system including a gas capturing unit, a gas dilution unit, a test sample storage unit, a gas sensor unit, and a data analysis unit, according to an embodiment of the present disclosure.

FIG. 3 is a schematic diagram of a gas sensor system including a gas capturing unit, a gas concentration unit, a gas sensor unit, and a data analysis unit, according to an embodiment of the present disclosure.

FIG. 4 is a schematic diagram of a gas sensor system including a gas capturing unit, a gas concentration unit, a test sample storage unit, a gas sensor unit, and a data analysis unit, according to an embodiment of the present disclosure.

FIG. 5 is a graph illustrating results measured for NH₃, H₂, and Acetone gases at a temperature of 300° C. using ZnO, Au—ZnO, and Pd—ZnO sensors, according to an embodiment of the present disclosure. The graph shows the correlation between K and LLOD, the correlation between β (Beta) and LLOD, and the correlation between n and LLOD.

FIG. 6 is a graph illustrating results measured for NH₃, H₂, and Acetone gases at a temperature of 350° C. using ZnO, Au—ZnO, and Pd—ZnO sensors, according to an embodiment of the present disclosure. The graph shows the correlation between K and LLOD, the correlation between β (Beta) and LLOD, and the correlation between n and LLOD.

FIG. 7 is a graph illustrating results measured for NH₃, H₂, and Acetone gases at a temperature of 400° C. using ZnO, Au—ZnO, and Pd—ZnO sensors, according to an embodiment of the present disclosure. The graph shows the correlation between K and LLOD, the correlation between β (Beta) and LLOD, and the correlation between n and LLOD.

FIG. 8 is a graph illustrating results measured for NH₃, H₂, and Acetone gases at a temperature of 450° C. using ZnO, Au—ZnO, and Pd—ZnO sensors, according to an embodiment of the present disclosure. The graph shows the correlation between K and LLOD, the correlation between β (Beta) and LLOD, and the correlation between n and LLOD.

### DETAILED DESCRIPTION

Hereinafter, the present disclosure will be described in detail. In describing the present disclosure, detailed descriptions of related known configurations or functions may be omitted.

Terms or words used in the present specification and claims should not be construed as being limited to common or dictionary meanings, but should be construed with meanings and concepts consistent with the technical aspects of the present disclosure.

The embodiments disclosed in the present specification and the configurations shown in the drawings are preferred embodiments of the present disclosure, and since they do not represent all of the technical ideas of the present disclosure, there may be various equivalents and modified examples capable of replacing them at the time of filing the present application.

The present disclosure provides a gas sensing method including: i) capturing an external gas; ii) measuring a resistance value (Rₒ) of a gas sensor under a base gas condition for stabilizing the gas sensor; iii) changing a concentration of the external gas and measuring resistance values (R_{g}) of the gas sensor under two or more concentration conditions with different concentrations; iv) calculating ratios (R_{g}/Rₒ) of the resistance value of the gas sensor under the base gas condition to the resistance values of the gas sensor under the two or more concentration conditions with different concentrations, and then deriving two or more kinetic parameters or static parameters from the ratios (R_{g}/Rₒ); and v) determining a type of the gas by comparing the correlation of the two or more kinetic parameters or static parameters calculated in the operation iv) with a standard correlation of two or more kinetic parameters or static parameters.

In the method, for the capturing of the external gas in the operation i), both active capturing using a pump or passive capturing of gas naturally through diffusion or the like may be used.

The external gas may be captured using various gas capturing devices known in the art.

In one embodiment, the gas capturing device may be composed only of a container for storing the captured gas, and may further include one or more selected from the group consisting of a filter for filtering out large particles such as dust, an adsorbent for selectively adsorbing a specific gas, a moisture removal filter for adsorbing moisture to minimize the influence of moisture in the air, a pump for moving the gas to a capturing container, a diffusion tube for diffusing the gas, a vacuum device for creating a vacuum state, and a device for analyzing and monitoring the external gas.

In the method, in the operation ii), the base gas may be air, an inert gas such as nitrogen or argon, or a mixed gas of oxygen and an inert gas, and a gas of the same or different type as a diluent gas may be used.

The operation ii) may be performed to measure R₀, the resistance value of the gas sensor in the reference state.

In the method, in the operation iii), the adjustment of the concentration of the captured external gas may be performed by varying a mixing ratio of the captured external gas and a diluent gas (for example, air, an inert gas such as nitrogen or argon, or a mixed gas of oxygen and an inert gas), or by applying pressure to the captured external gas to vary a degree of concentration.

The concentration may be adjusted to various desired concentrations by preparing them using various gas dilution devices or gas concentration devices known in the art.

In one embodiment, the gas dilution device may include a container for storing a diluent gas and a diluent gas injection unit for injecting the diluent gas into the captured external gas, and may further include one or more selected from the group consisting of a plurality of storage containers for storing two or more test samples with different concentrations created by injecting the diluent gas into the captured external gas, a device for controlling a gas (captured gas and diluted gas) flow velocity, a device for detecting a gas leak, a flow rate control device, and a real-time monitoring and control device.

In one embodiment, the gas concentration device may include a compressor for increasing a pressure of a gas by reducing a volume of the gas, and may further include one or more selected from the group consisting of a plurality of storage containers for storing two or more test samples concentrated to have different concentrations, a device for measuring a pressure of the container, a pressure relief valve for maintaining a normal value by releasing pressure so that the pressure does not exceed a specified value, a device for detecting overpressure and leakage, and a real-time monitoring and control device.

In the method, in the operation iii), the various concentration conditions may be created in a chamber including the gas sensor, or test samples with different concentration conditions may be prepared before being exposed to the gas sensor, stored in a storage container, and then moved to the chamber including the gas sensor.

In one embodiment, when the various concentration conditions are created in the chamber including the gas sensor, a gas capturing device and a gas dilution device are respectively connected to the chamber including the gas sensor, and the captured gas and a diluent gas may be injected into the chamber in appropriate amounts by a flow rate control device to adjust the concentration and create various concentration conditions.

In one embodiment, when the various concentration conditions are created in the chamber including the gas sensor, first, a gas capturing device and a gas concentration device are respectively connected to the chamber including the gas sensor, and the captured gas is injected into the chamber in an appropriate amount by a flow rate control device. Then, pressure is applied to the chamber by the gas concentration device to adjust the concentration and create various concentration conditions.

In one embodiment, when the various concentration conditions are created outside the chamber before being exposed to the gas sensor, first, a gas capturing device and a gas dilution device are respectively connected to a plurality of storage containers, and a captured gas and a diluent gas are injected into the plurality of storage containers in appropriate amounts by a flow rate control device to adjust the concentration and prepare test samples with different concentrations. Then, the test samples with different concentrations may be moved from the plurality of storage containers to the chamber including the gas sensor to create various concentration conditions.

In one embodiment, when the various concentration conditions are created outside the chamber before being exposed to the gas sensor, first, a gas capturing device and a gas concentration device are respectively connected to a plurality of storage containers, and after a captured gas is injected into the plurality of storage containers, pressure is applied to the inside of the storage containers by the gas concentration device to adjust the concentration and prepare test samples with different concentrations. Then, the test samples with different concentrations may be moved from the plurality of storage containers to the chamber including the gas sensor to create various concentration conditions.

In the method, in the operation iii), separately from the concentration, one or two or more of temperature, humidity, or light absorbance may be varied to additionally create conditions with different temperatures, humidity, or light absorbances. Through this, by preparing test samples of more various combinations by varying other conditions in addition to the concentration and performing the gas sensing of the present disclosure, the gas may be distinguished more accurately as the result value due to the change in other conditions is added, compared to distinguishing the gas with the result value due to the concentration change. As a result, the selectivity for the gas may be improved.

The conditions with different temperatures, humidity, or light absorbances may be controlled using a temperature control device, a humidity control device, and a light source device, respectively. At this time, the temperature control device, the humidity control device, or the light source device may be a separate device from the chamber including the gas sensor and may be provided outside the chamber. Alternatively, the temperature control device, the humidity control device, or the light source device may be provided inside the chamber including the gas sensor, such as a Micro-Electro-Mechanical System (MEMS), and integrated into a single system.

In one embodiment, the temperature may be treated differently using a temperature control device (for example, a heater or a cooler). Furthermore, the humidity may be treated differently in terms of moisture supply amount using a humidity control device (for example, a moisture supplier or a humidity generator). Furthermore, the light absorbance may be treated differently in terms of wavelength or intensity of light using a light source device (for example, a UV light emitting device, a UV-LED light emitting device, or a visible light wavelength LED light emitting device). Various test samples in which one or two or more of the temperature, humidity, or light absorbance are different may be prepared.

In the method, in the operation ii) or operation iii), when the gas sensor is exposed to a base gas or an external gas, a relative change in resistance value for each concentration of the gas may be derived by measuring the resistance value of the sensor.

The gas sensor may be a semiconductor-type gas sensor based on a metal oxide material (for example, a metal oxide selected from the group consisting of ZnO, SnO₂, In₂, O₃, WO₃, CuO, and NiO) or a metal-metal oxide material (for example, a material in which metal particles selected from the group consisting of platinum (Pt), palladium (Pd), silver (Ag), and gold (Au) are attached to the metal oxide) whose resistance changes in response to a specific gas.

In the method, in the operation ii) or operation iii), the gas sensor may be included in a chamber, and multiple gas sensing may be performed by including a plurality of gas sensors for detecting different types of gases in the chamber.

In one embodiment, the gas sensor may further include one or more of a power supply, a source measure unit (SMU) for precisely measuring a source voltage or current, or device connection accessories, in addition to the material whose resistance changes in response to a specific gas.

In one embodiment, the gas sensor may be located together with one or more of the temperature control device, the humidity control device, or the light source device inside the chamber to measure a resistance value by varying the temperature, humidity, or light absorbance of the gas.

In the method, in the operation iv), the kinetic or static parameters may be derived by being directly calculated using a calculation formula known in the art, such as a power law model, from the change in the resistance values of the gas sensor for each concentration.

Furthermore, the kinetic or static parameters may be derived through a data processing program performed with output data of the gas sensor (e.g., time-concentration data of resistance/current/voltage signals) as input. The data processing program may be one or two or more programs selected from the group including OriginPro, MATLAB, Python, LabVIEW, Excel, and R. The program may, for example, extract a regression, curve fitting, and slope/exponent value of Equation 1 below.

The kinetic parameter may be one selected from the group consisting of K, β (Beta), LLOD (lower limit of detection), Eb (Energy barrier), and n (Power law Exponent), and the static parameter may be one selected from the group consisting of t90, T90, carrier concentration, and oxygen vacancy.

The kinetic parameter or static parameter may be defined as follows.

K: K is a reaction rate coefficient derived by mathematically analyzing the resistance response behavior of the gas sensor. K is a value calculated by a formula, is related to the adsorption and desorption of oxygen, and is a dynamic parameter related to the rate at which an external gas reacts with oxygen and the material surface.

β (beta): β is an exponential value representing the relationship between the sensor response and the gas concentration. β is a value calculated by a formula and is a dynamic parameter related to the interaction between an external gas and the type of ionized oxygen present on the material surface.

LLOD: LLOD is the minimum gas concentration at which a gas may be detected. LLOD is a gas concentration at which an external gas may be detected and is a dynamic parameter obtained from K and β (beta), which are calculated by a formula.

T90: T90 is the gas detection speed, the time required for the sensor signal to reach 90% of the final response value after the injection of an external gas. T90 is a static parameter that represents the response time and is a numerical value indicating how quickly an external gas is detected.

t90: t90 is a recovery characteristic of the sensor, the time required for the sensor signal to recover to 90% of its initial state after the gas is removed. t90 is a static parameter that represents the recovery time and is a numerical value indicating how quickly an external gas is recovered.

Carrier Concentration: Carrier concentration is the density of free charges (electrons or holes) contributing to electrical conduction within a semiconductor material. The carrier concentration is a material parameter related to various dynamic parameters and static parameters.

Oxygen vacancy: An oxygen vacancy is a point defect formed by the absence of an oxygen atom in an oxide crystal structure. The oxygen vacancy is a material parameter related to various dynamic parameters and static parameters.

Eb: Eb is the height of the energy barrier in the electron depletion layer formed by oxygen adsorbed on the surface of the material. Eb is a dynamic parameter.

n: n signifies the form of charged oxygen species formed when adsorbed oxygen molecules on a solid surface or interface capture or release electrons. n is a value determining the type of ionized oxygen ion species present on the material surface and is a dynamic parameter.

In one embodiment, K, β (Beta), and LLOD may be derived by Equation 1 below.

$$Sensing response ( S ) = \frac{{R}_{g}}{{R}_{a}} = K \cdot {C}^{β} \qquad Equation 1$$

where,
R_{g}: electrical resistance when exposed to an external gas;
Rₐ: electrical resistance when exposed to a base gas; and
C: concentration of the external gas.
In one embodiment, Eb may be derived by Equation 2 below.

$${R}_{e} = \frac{{R}_{g}}{{R}_{0}} = \frac{{N}_{d}}{{n}_{s}} = exp ( \frac{{qV}_{s}}{kT} ) \qquad Equation 2$$

$$qVs = {E}_{b} = \frac{{q}^{2}}{2 ε} \frac{{n}_{s}^{2}}{{N}_{o}} = \frac{{q}^{2}}{2 ε} {N}_{o} {W}^{2} = \frac{{k}_{B} T}{2} \frac{{W}^{2}}{{λ}_{D}^{2}}$$

where,
R_{g}: resistance in a gas atmosphere;
Rₒ: resistance in a flat band state;
Rₑ: reduced sensor resistance;
Vₛ: potential energy of electrons;
q: Coulomb's charge;
ε: dielectric constant;
Ns: surface charge carrier concentration;
No: bulk carrier concentration; and
Nd: donor density.

In the method, the operation v) is to distinguish a specific type of gas by comparing the correlation of the two or more kinetic parameters or static parameters calculated in the operation iv) with a pre-established standard correlation of two or more kinetic parameters or static parameters for a specific type of gas.

In the operation v), the correlation of the two or more kinetic parameters or static parameters is a meaning that includes all correlations among the two or more kinetic or static parameters, and in particular, may be one or more selected from the group consisting of the correlation between K and LLOD, the correlation between β (Beta) and LLOD, and the correlation between n and LLOD.

In the operation v), the standard correlation of the two kinetic parameters or static parameters may be data obtained by repeating the operations of i) to v) according to the present disclosure multiple times for a target gas to be measured and calculating the correlation of the two or more kinetic or static parameters of the target gas.

In the operation v), the correlation of the two or more kinetic parameters or static parameters may be visualized as a scatter plot, and at this time, it may be compared with a standard scatter plot visualizing the standard correlation of the two or more kinetic parameters or static parameters.

The present disclosure utilizes a mechanism in which the electrical resistance value changes according to the characteristics of a specific material of a gas sensor, and applies the mechanism by which a reaction occurs on the surface of the material. In particular, a quantitative numerical value was calculated from the resistance change width occurring when an external gas is flowed under the same environmental conditions.

In one embodiment of the present disclosure, it was confirmed that the characteristics of the material of the gas sensor may affect the reactivity, thereby exhibiting a difference in the change in electrical resistance. For example, for a specific material, the resistance value increases or decreases depending on the interaction with gas molecules, due to the electron transfer mechanism or ion adsorption characteristics on the material surface. This resistance change may vary depending on external environmental conditions, the type and concentration of the gas, and the microstructural characteristics of the material. The resistance change width occurring when an external gas is flowed may be calculated by the following method. Under the same environmental conditions, a resistance value in a reference state is set to R₀, and a resistance value in a state where gas is flowed is measured as R_{g}. The resistance change ratio may be defined by the following equation.

$$Δ R = \frac{{R}_{g} - {R}_{o}}{{R}_{o}} \times 100 ( \% ) \qquad Equation 3$$

It may be confirmed that the resistance value obtained through the above equation shows a different resistance value change width depending on the material characteristics of the gas sensor, and through this, the sensitivity and reactivity of the material of the gas sensor may be evaluated.

In one embodiment of the present disclosure, the change in resistance value according to the type and concentration of an external gas was experimentally confirmed for various materials of a gas sensor. For example, when the gas sensor is a metal oxide material, the change width of the resistance value was larger for a specific gas, which may be seen as showing a different mechanism due to the electron transfer effect according to surface adsorption depending on the material characteristics of the gas sensor.

Furthermore, the present disclosure provides a gas sensing system for performing the gas sensing method described above.

The gas sensor system is a gas sensor system for performing the gas sensing method among the gas sensing methods according to the present disclosure, which is applied by preparing external gas at various concentrations through gas dilution.

The gas sensing system of the present disclosure includes: a gas capturing unit for capturing an external gas; a gas dilution unit for storing and injecting a diluent gas; a gas sensor unit for measuring a resistance value (Rₒ) of the gas sensor under a base gas condition for stabilizing the gas sensor, and resistance values (R_{g}) of the gas sensor under two or more concentration conditions with different concentrations by changing a concentration of the external gas by the gas dilution unit; and a data analysis unit for comparing and analyzing the correlation of two or more kinetic parameters or static parameters calculated from the resistance values of the gas sensor unit.

Here, the data analysis unit first calculates ratios (R_{g}/Rₒ) of the resistance value (Rₒ) of the gas sensor under the base gas condition to the resistance values (R_{g}) of the gas sensor under the two or more concentration conditions with different concentrations from the resistance values of the gas sensor unit. Then, two or more kinetic or static parameters are derived from the ratios (R_{g}/Rₒ). Then, the correlation of the two or more kinetic or static parameters is compared and analyzed with a standard correlation of two or more kinetic parameters or static parameters.

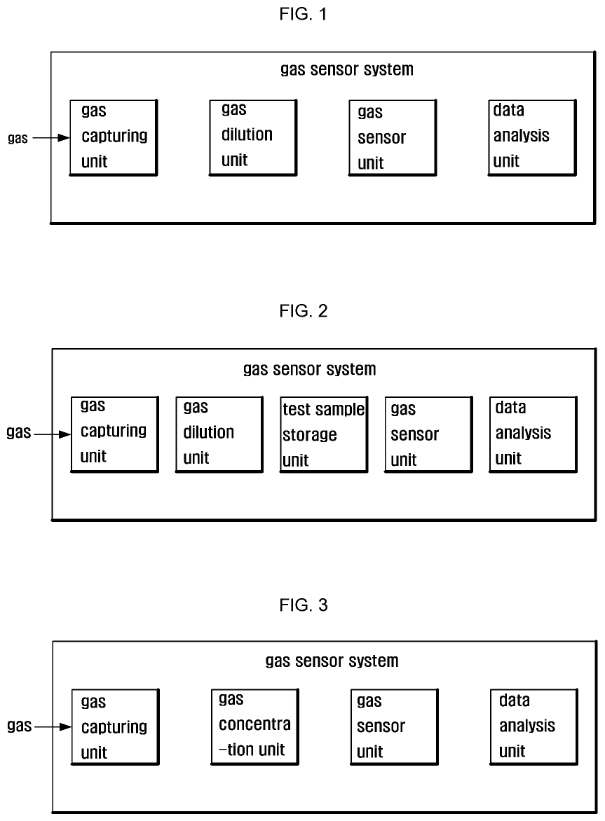

The gas sensor system may be arranged as shown in the schematic diagrams disclosed in FIG. 1 and FIG. 2.

FIG. 1 is a gas sensor system composed of a gas capturing unit, a gas dilution unit, a gas sensor unit, and a data analysis unit.

FIG. 2 is a gas sensor system further including a test sample storage unit in FIG. 1.

In the gas sensor system, the gas capturing unit is a means for capturing an external gas, and at this time, the capturing of the external gas may be either active capturing using a pump or passive capturing of gas naturally through diffusion or the like.

The gas capturing unit may use various gas capturing devices known in the art.

In one embodiment, the gas capturing unit may be composed only of a container for storing the captured gas, and may further include one or more selected from the group consisting of a filter for filtering out large particles such as dust, an adsorbent for selectively adsorbing a specific gas, a moisture removal filter for adsorbing moisture to minimize the influence of moisture in the air, a pump for moving the gas to a capturing container, a diffusion tube for diffusing the gas, a vacuum device for creating a vacuum state, and a device for analyzing and monitoring the external gas.

In the gas sensor system, the gas dilution unit is a means for injecting a diluent gas (for example, air, an inert gas such as nitrogen or argon, or a mixed gas of oxygen and an inert gas) into the captured external gas at different supply rates, and the concentration of the external gas may be prepared at various concentrations using various gas dilution devices known in the art.

In one embodiment, the gas dilution unit may include a container for storing the diluent gas and a diluent gas injection unit for injecting a diluent gas into the captured external gas, and may further include one or more selected from the group consisting of a device for controlling a gas flow velocity, a device for detecting a gas leak, a flow rate control device, and a real-time monitoring and control device.

In the gas sensor system, the gas sensor unit is a means for measuring the resistance value of the sensor when exposed to a base gas or an external gas, and through this, a relative change in resistance value for each concentration of the gas may be derived.

The gas sensor unit may be composed of a gas sensor and a chamber including the gas sensor.

The changing of the concentration may be performed using the gas dilution unit. The changing of the concentration may be created in the chamber including the gas sensor, or test samples with changed concentrations may be prepared before being exposed to the gas sensor, stored in a storage container, and then moved to the chamber including the gas sensor.

In one embodiment, when created in the chamber including the gas sensor, a gas capturing device and a gas dilution device may be respectively connected to the chamber including the gas sensor, and the captured gas and a diluent gas may be injected into the chamber in appropriate amounts by a flow rate control device to adjust the concentration.

In one embodiment, when created outside the chamber before being exposed to the gas sensor, first, a gas capturing device and a gas dilution device are respectively connected to a plurality of storage containers, and a captured gas and a diluent gas are injected into the plurality of storage containers in appropriate amounts by a flow rate control device to adjust the concentration and prepare test samples with different concentrations. Then, the test samples with different concentrations may be moved from the plurality of storage containers to the chamber including the gas sensor.

In the gas sensor unit, the gas sensor may be a semiconductor-type gas sensor based on a metal oxide material (for example, a metal oxide selected from the group consisting of ZnO, SnO₂, In₂, O₃, WO₃, CuO, and NiO) or a metal-metal oxide material (for example, a material in which metal particles selected from the group consisting of platinum (Pt), palladium (Pd), silver (Ag), and gold (Au) are attached to the metal oxide) whose resistance changes in response to a specific gas.

The gas sensor unit may include one gas sensor in the chamber, and may include a plurality of gas sensors in the chamber to perform multiple gas sensing for detecting different types of gases.

The gas sensor unit may further include one or more of a power supply, a source measure unit (SMU) for precisely measuring a source voltage or current, or device connection accessories, in addition to the above-mentioned material whose resistance changes in response to a specific gas.

The gas sensor unit may include one or more of a temperature control device, a humidity control device, or a light source device therein to measure a resistance value by varying the temperature, humidity, or light absorbance of the gas.

For example, the temperature control device may be a heater or a cooler. The humidity control device may be a moisture supplier or a humidity generator (for example, an ultrasonic humidifier, a heating humidifier, a composite humidifier, etc.). The light source device may be a UV light emitting device, a UV-LED light emitting device, or a visible light wavelength LED light emitting device.

In the gas sensor system, a test sample storage unit may be included separately from the gas sensor unit.

The test sample storage unit may be a means for respectively storing the two or more test samples with different concentrations prepared in the gas dilution unit.

In one embodiment, the test sample storage unit may include two or more plural containers for storing two or more test samples, and may further include one or more selected from the group consisting of a flow velocity and flow rate control device, a device for detecting a gas leak, and a real-time monitoring and control device.

In the gas sensor system, one or more of a temperature control device, a humidity control device, or a light source device may be further included externally, separate from the gas sensor unit.

The temperature control device, the humidity control device, or the light source device may additionally prepare test samples with different temperatures, humidity, or light absorbances by varying one or two or more of temperature, humidity, or light absorbance separately from the concentration. Through this, by preparing test samples of more various combinations by varying other conditions in addition to the concentration and operating the gas sensor system of the present disclosure, the gas may be distinguished more accurately as the result value due to the change in other conditions is added, compared to distinguishing the gas with the result value due to the concentration change. As a result, the selectivity for the gas may be improved.

In the gas sensor system, the data analysis unit first calculates a change in resistance value for each concentration from the resistance value calculated by the gas sensor unit. Then, two or more kinetic or static parameters are derived from the change in the resistance value using a calculation formula known in the art, such as a power law model. Then, it is compared with a pre-established standard correlation of two kinetic parameters or static parameters for a specific type of gas.

The data analysis unit may use one or more data processing programs selected from the group consisting of OriginPro, MATLAB, Python, LabVIEW, Excel, and R.

The kinetic parameter may be one selected from the group consisting of K, β (Beta), LLOD (lower limit of detection), Eb (Energy barrier), and n (Power law Exponent), and the static parameter may be one selected from the group consisting of t90, T90, carrier concentration, and oxygen vacancy.

The kinetic parameter or static parameter may be defined as follows.

K: K is a reaction rate coefficient derived by mathematically analyzing the resistance response behavior of the gas sensor. K is a value calculated by a formula, is related to the adsorption and desorption of oxygen, and is a dynamic parameter related to the rate at which an external gas reacts with oxygen and the material surface.

β (beta): β is an exponential value representing the relationship between the sensor response and the gas concentration. β is a value calculated by a formula and is a dynamic parameter related to the interaction between an external gas and the type of ionized oxygen present on the material surface.

LLOD: LLOD is the minimum gas concentration at which a gas may be detected. LLOD is a gas concentration at which an external gas may be detected and is a dynamic parameter obtained from K and β (beta), which are calculated by a formula.

T90: T90 is the gas detection speed, which is the time required for the sensor signal to reach 90% of the final response value after the injection of an external gas. T90 is a static parameter that represents the response time and is a numerical value indicating how quickly an external gas is detected.

t90: t90 is a recovery characteristic of the sensor, which is the time required for the sensor signal to recover to 90% of its initial state after the gas is removed. t90 is a static parameter that represents the recovery time and is a numerical value indicating how quickly an external gas is recovered.

Carrier Concentration: Carrier concentration is the density of free charges (electrons or holes) contributing to electrical conduction within a semiconductor material. The carrier concentration is a material parameter related to various dynamic parameters and static parameters.

Oxygen vacancy: An oxygen vacancy is a point defect formed by the absence of an oxygen atom in an oxide crystal structure. The oxygen vacancy is a material parameter related to various dynamic parameters and static parameters.

Eb: Eb is the height of the energy barrier in the electron depletion layer formed by oxygen adsorbed on the surface of the material. Eb is a dynamic parameter.

n: n signifies the form of charged oxygen species formed when adsorbed oxygen molecules on a solid surface or interface capture or release electrons. n is a value determining the type of ionized oxygen ion species present on the material surface and is a dynamic parameter.

In one embodiment, K, β (Beta), and LLOD may be derived by Equation 1 below.

$$Sensing response ( S ) = \frac{{R}_{g}}{{R}_{a}} = K \cdot {C}^{β} \qquad Equation 1$$

where,
Rg: electrical resistance when exposed to an external gas;
Ro: electrical resistance when exposed to a base gas; and
C: concentration of the external gas.

In one embodiment, Eb may be derived by Equation 2 below.

$${R}_{e} = \frac{{R}_{g}}{{R}_{0}} = \frac{{N}_{d}}{{n}_{s}} = exp ( \frac{{qV}_{s}}{kT} ) \qquad Equation 2$$

$$qVs = {E}_{b} = \frac{{q}^{2}}{2 ε} \frac{{n}_{s}^{2}}{{N}_{o}} = \frac{{q}^{2}}{2 ε} {N}_{o} {W}^{2} = \frac{{k}_{B} T}{2} \frac{{W}^{2}}{{λ}_{D}^{2}}$$

where,
Rg: resistance in a gas atmosphere;
Ro: resistance in a flat band state;
Re: reduced sensor resistance;
Vs: potential energy of electrons;
q: Coulomb's charge;
ε: dielectric constant;
Ns: surface charge carrier concentration;
No: bulk carrier concentration; and
Nd: donor density.

The correlation of the two or more kinetic parameters or static parameters is a meaning that includes all correlations among the two or more kinetic or static parameters, and in particular, may be one or more selected from the group consisting of the correlation between K and LLOD, the correlation between β (Beta) and LLOD, and the correlation between n and LLOD.

The standard correlation of the kinetic parameters or static parameters may be data obtained by repeating the process through the gas sensor according to the present disclosure multiple times for a target gas and calculating the correlation of the two or more kinetic parameters or static parameters of the target gas.

The correlation of the two or more kinetic parameters or static parameters may be visualized as a scatter plot, and at this time, it may be compared with a standard scatter plot visualizing the standard correlation of the two or more kinetic parameters or static parameters.

Furthermore, the present disclosure provides another type of gas sensing system for performing the gas sensing method described above.

The gas sensor system is a gas sensor system for performing the gas sensing method among the gas sensing methods according to the present disclosure, which is applied by preparing an external gas at various concentrations through gas concentration.

The gas sensor system includes: a gas capturing unit for capturing an external gas; a gas concentration unit for compressing the captured external gas; a gas sensor unit for measuring a resistance value (Rₒ) of the gas sensor under a base gas condition for stabilizing the gas sensor, and resistance values (R_{g}) of the gas sensor under two or more concentration conditions with different concentrations by changing a concentration of the external gas by the gas concentration unit; and a data analysis unit for comparing and analyzing a correlation of two or more kinetic parameters or static parameters calculated from the resistance values of the gas sensor unit.

Here, the data analysis unit first calculates ratios (R_{g}/Rₒ) of the resistance value (Rₒ) of the gas sensor under the base gas condition to the resistance values (R_{g}) of the gas sensor under the two or more concentration conditions with different concentrations from the resistance values of the gas sensor unit. Then, two or more kinetic or static parameters are derived from the ratios (R_{g}/Rₒ). Then, the correlation of the two or more kinetic or static parameters is compared and analyzed with a standard correlation of two or more kinetic parameters or static parameters.

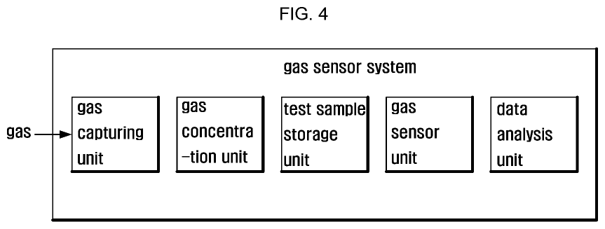

The gas sensor system may be configured as shown in the schematic diagrams disclosed in FIG. 3 and FIG. 4.

FIG. 3 is a gas sensor system composed of a gas capturing unit, a gas concentration unit, a gas sensor unit, and a data analysis unit.

FIG. 4 is a gas sensor system further including a test sample storage unit in FIG. 3.

In the gas sensor system, the descriptions of each component of the gas capturing unit, the gas sensor unit, and the data analysis unit are the same as those of the gas sensor system described above.

In the gas sensor system, the gas concentration unit is a means for changing the concentration by reducing the volume of the gas by applying pressure to the captured external gas, and various gas concentration devices known in the art may be used.

In one embodiment, the gas concentration unit may include a compressor for increasing the pressure of a gas by reducing the volume of the gas, and may further include one or more selected from the group consisting of a device for measuring the pressure of the container, a pressure relief valve for maintaining a normal value by releasing pressure so that the pressure does not exceed a specified value, a device for detecting overpressure and leakage, and a real-time monitoring and control device.

The changing of the concentration may be performed using the gas concentration unit. The changing of the concentration may be created in the chamber including the gas sensor, or test samples with different concentrations may be prepared before being exposed to the gas sensor, stored in a storage container, and then moved to the chamber including the gas sensor.

In one embodiment, when created in the chamber including the gas sensor, a gas capturing device and a gas concentration device may be respectively connected to the chamber including the gas sensor, and after the captured gas is injected into the chamber in an appropriate amount by a flow rate control device, pressure may be applied to the chamber by the gas concentration device to adjust the concentration.

In one embodiment, when created outside the chamber before being exposed to the gas sensor, first, a gas capturing device and a gas concentration device are respectively connected to a plurality of storage containers, and after a captured gas is injected into the plurality of storage containers, pressure is applied to the inside of the storage containers by the gas concentration device to adjust the concentration and prepare test samples with different concentrations. Then, the test samples with different concentrations may be moved from the plurality of storage containers to the chamber including the gas sensor.

In the gas sensor system, a test sample storage unit may be included separately from the gas sensor unit.

The test sample storage unit may be a means for respectively storing two or more test samples with different concentrations prepared in the gas concentration unit.

In one embodiment, the test sample storage unit may include a plurality of containers for storing two or more test samples, and may further include one or more selected from the group consisting of a flow velocity and flow rate control device, a device for detecting a gas leak, and a real-time monitoring and control device.

In the gas sensor system, one or more of a temperature control device, a humidity control device, or a light source device may be further included externally, separate from the gas sensor unit.

The temperature control device, the humidity control device, or the light source device may additionally prepare test samples with different temperatures, humidity, or light absorbances by varying one or two or more of temperature, humidity, or light absorbance separately from the concentration. Through this, by preparing test samples of more various combinations by varying other conditions in addition to the concentration and operating the gas sensor system of the present disclosure, the gas may be distinguished more accurately as the result value due to the change in other conditions is added, compared to distinguishing the gas with the result value due to the concentration change. As a result, the selectivity for the gas may be improved.

In addition, the present disclosure provides an electronic device including the gas sensor system according to the present disclosure.

The gas sensor system may be miniaturized, and through this, it may be easily integrated into various everyday electronic devices such as smart watches, attachable sensors, and portable electronic devices.

Hereinafter, the present disclosure will be described in more detail through examples.

These examples are only for explaining the present disclosure more specifically, and it is apparent to those skilled in the art that the scope of the present disclosure is not limited by these examples according to the gist of the present disclosure.

### Example 1 Fabrication of Gas Sensor

For the gas sensor device, a pre-fabricated MEMS system (Maimosi) device was purchased, the components were assembled, and then a gas sensing material was applied on a micrometer scale onto the assembled gas sensor device by drop-casting or spraying so that the electrodes were covered. The resistance of the gas sensing material placed on the electrodes changes depending on the atmosphere, temperature, humidity, and wavelength and intensity of the light source in the gas sensing chamber. At this time, for the gas sensing materials (ZnO, Au—ZnO, and Pd—ZnO), ZnO, Au—ZnO, and Pd—ZnO, which are oxide semiconductors with excellent reactivity with surface oxygen ions and gases due to the formation of a surface defect structure through heat treatment, were used respectively.

### Example 2 Derivation of Graphs Through Gas Sensor

The gas atmosphere was controlled by connecting O₂/N₂ mixed gas with 21% O₂ and NH₃, Acetone, H₂ gas cylinders to a Mass Flow Controller (MFC). The gas was ordered to match a concentration of 100 ppm/N₂ balance (Ilsung Gas), a regulator was connected to the gas cylinder to check the pressure and amount of the gas, and the flow rate of the gas was controlled through the Mass Flow Controller (MFC) and flowed into the gas sensing chamber to form a gas atmosphere. The detailed concentration and flow rate were controlled through a gas sensor tester (YNI-FLOW&PRESSURE CONTROLLER: MR-5000) and a built-in program. The temperature of the gas sensing chamber and the gas sensor device was controlled through a temperature controller (MSTECH—Hot Chuck Controller: MST-1000H). The performance of the gas sensor was confirmed by simultaneously measuring voltage, current, and resistance through a Keithley instrument (2450 Sourcemeter). The gas sensing performance was determined by measuring how much the resistance changed when a gas was flowed compared to the resistance when an O₂/N₂ mixed gas was flowed, and a larger change in resistance even at a low concentration was judged as a better gas sensing material.

During the gas sensing experiment, an O₂/N₂ mixed gas was flowed over the fabricated gas sensor to adsorb oxygen on the surface of the gas sensor material, and then it was stabilized for 30 minutes or more until there was no change in the resistance of the sensor. After that, a desired target gas (NH₃, Acetone, H₂, etc.) was flowed, the gas sensor material and the gas reacted, the surface charge concentration of the material changed, and then the resistance change over time was observed.

The ammonia (NH₃) gas was set in the order of 0.1 ppm, 0.25 ppm, 0.5 ppm, 1 ppm, 5 ppm, 25 ppm, and 50 ppm for each concentration, and measurement was performed for 15 minutes for each concentration.

The acetone (Acetone) gas was set in the order of 0.75 ppm, 2.5 ppm, 10 ppm, 50 ppm, 100 ppm, 250 ppm, and 275 ppm for each concentration, and measurement was performed for 15 minutes for each concentration.

The hydrogen (H₂) gas was set in the order of 0.75 ppm, 1 ppm, 2.5 ppm, 5 ppm, 10 ppm, 25 ppm, and 50 ppm for each concentration, and measurement was performed for 15 minutes for each concentration.

In the above experiment, to achieve each target concentration, the mixing ratio of the target gas and air was precisely adjusted using a Mass Flow Controller (MFC). Through this, the gas environment according to the target concentration was accurately implemented, and the measurement data at each concentration was maintained for a fixed time to obtain reliable results.

The response of the gas sensor was obtained through the following equation.

$$( Resistance when gas is flowed ) \div ( Resistance when {O}_{2} / {N}_{2} mixed gas is flowed ) \qquad Equation$$

After obtaining the response of the gas sensor, K, Beta, and LLOD were calculated through a power law calculation formula.

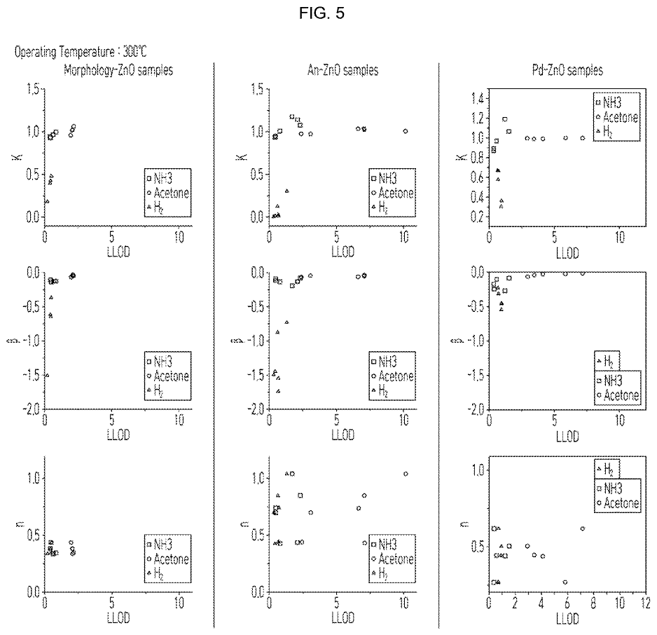

The results of graphing the data from three repeated measurements of the correlation between K and LLOD, the correlation between β (Beta) and LLOD, and the correlation between n and LLOD for each temperature (300° C., 350° C., 400° C., and 450° C.) for NH₃, H₂, and Acetone as a scatter plot are shown in FIG. 5 to FIG. 8.

FIG. 5 is a scatter plot of data from three repeated measurements of the correlation of K&LLOD, Beta&LLOD, and n&LLOD parameters for NH₃, H₂, and Acetone gases at 300° C. using ZnO, Au—ZnO, and Pd—ZnO sensors. As shown in FIG. 5, although there were slight differences for each sensor, the distribution of points for each gas formed a certain pattern, such as being clustered in a certain coordinate group or forming a line. It was confirmed that this pattern of FIG. 5 was formed differently for each gas.

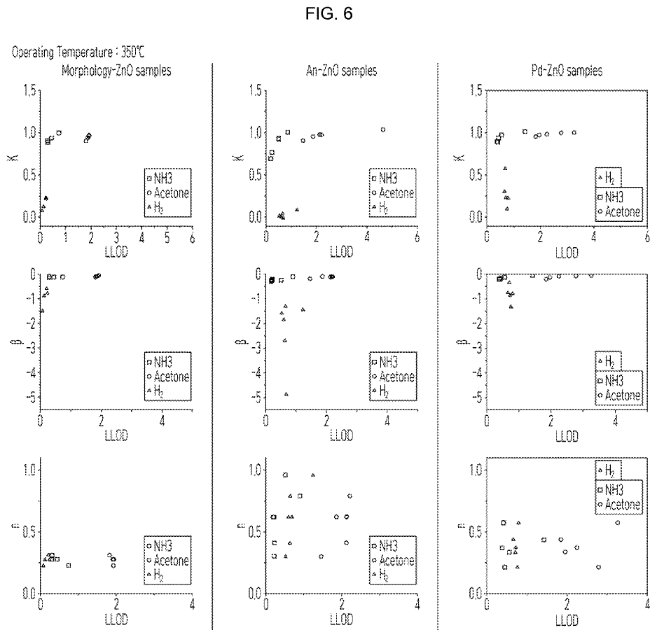

FIG. 6 is a scatter plot of data from three repeated measurements of the correlation of K&LLOD, Beta&LLOD, and n&LLOD parameters for NH₃, H₂, and Acetone gases at 350° C. using ZnO, Au—ZnO, and Pd—ZnO sensors. As shown in FIG. 6, although there were slight differences for each sensor, the distribution of points for each gas formed a certain pattern, such as being clustered in a certain coordinate group or forming a line. This pattern of FIG. 6 was formed differently for each gas. It was confirmed that compared to FIG. 5, although the position on the coordinates was different, a similar pattern of point distribution was formed.

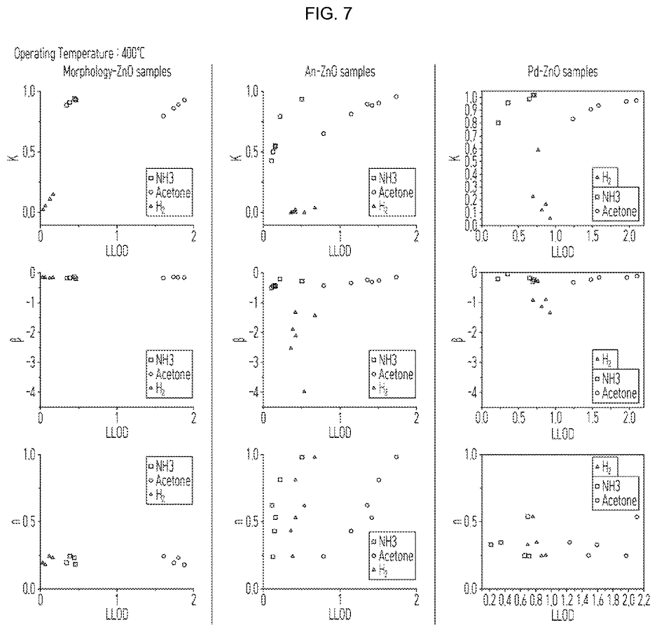

FIG. 7 is a scatter plot of data from three repeated measurements of the correlation of K&LLOD, Beta&LLOD, and n&LLOD parameters for NH₃, H₂, and Acetone gases at 400° C. in each of the ZnO, Au—ZnO, and Pd—ZnO sensors. As shown in FIG. 7, although there were slight differences for each sensor, the distribution of points for each gas formed a certain pattern, such as being clustered in a certain coordinate group or forming a line. This pattern of FIG. 7 was formed differently for each gas. It was confirmed that compared to FIG. 5 and FIG. 6, although the position on the coordinates was different, a similar pattern of point distribution was formed.

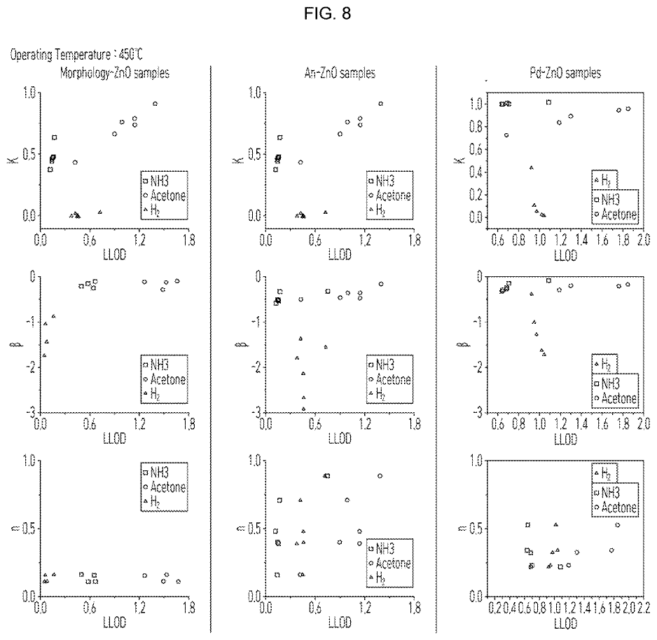

FIG. 8 is a scatter plot of data from three repeated measurements of the correlation of K&LLOD, Beta&LLOD, and n&LLOD parameters for NH₃, H₂, and Acetone gases at 450° C. using ZnO, Au—ZnO, and Pd—ZnO sensors. As shown in FIG. 8, although there were slight differences for each sensor, the distribution of points for each gas formed a certain pattern, such as being clustered in a certain coordinate group or forming a line. This pattern of FIG. 8 was formed differently for each gas. It was confirmed that compared to FIG. 5 to FIG. 7, although the position on the coordinates was different, a similar pattern of point distribution was formed.

Therefore, by establishing the correlation between K and LLOD, the correlation between β (Beta) and LLOD, and the correlation between n and LLOD for each type of gas (NH₃, H₂, and Acetone) according to FIG. 5 to FIG. 8 as a standard, and then comparing the results of measuring the correlation between K and LLOD, the correlation between § (Beta) and LLOD, and the correlation between n and LLOD for a future external gas with the correlation of the standard parameters, when mutually similar results are obtained, the type of gas (NH₃, H₂, and Acetone) may be distinguished.

What is claimed is:

1. A gas sensing method comprising: i) capturing an external gas; ii) measuring a resistance value (Rₒ) of a gas sensor under a base gas condition to stabilize the gas sensor; iii) changing a concentration of the external gas and measuring resistance values (R_{g}) of the gas sensor under two or more concentration conditions with different concentrations; iv) calculating ratios (R_{g}/Rₒ) of the resistance value of the gas sensor under the base gas condition to the resistance values of the gas sensor under the two or more concentration conditions with different concentrations, and then calculating two or more kinetic parameters or static parameters from the ratios (R_{g}/Rₒ); and v) determining a type of the gas by comparing the correlation of the two or more kinetic parameters or static parameters calculated in the operation iv) with a standard correlation of two or more kinetic parameters or static parameters.

2. The gas sensing method of claim 1, wherein in the operation iii), the two or more concentration conditions are created by varying a mixing ratio of the external gas and a diluent gas.

3. The gas sensing method of claim 2, wherein the diluent gas is any one selected from the group consisting of air, an inert gas, and a mixed gas of oxygen and an inert gas.

4. The gas sensing method of claim 1, wherein in the operation iii), the two or more concentration conditions are created by varying a degree of concentration for the external gas.

5. The gas sensing method of claim 1, wherein in the operation iii), the resistance values of the gas sensor are measured under a condition in which at least one selected from the group consisting of temperature, humidity, and light absorbance is different, by varying at least one selected from the group consisting of temperature, humidity, and light absorbance.

6. The gas sensing method of claim 5, wherein the temperature, the humidity, and the light absorbance are controlled using a temperature control device, a humidity control device, and a light source device disposed inside or outside a gas sensor unit, respectively.

7. The gas sensing method of claim 1, wherein in the operations ii) to iii), a gas sensor unit simultaneously measures resistance values for a plurality of gases using a plurality of gas sensors arranged to detect different gases, respectively.

8. The gas sensing method of claim 1, wherein in the operation iv), the kinetic parameter is one or more selected from the group consisting of K, β (Beta), LLOD (lower limit of detection), Eb (Energy barrier), and n (Power law Exponent), and the static parameter is one or more selected from the group consisting of t90, T90, carrier concentration, and oxygen vacancy.

9. The gas sensing method of claim 1, wherein in the operation v), the correlation of the kinetic parameters is one or more selected from the group consisting of a correlation between K and LLOD, a correlation between β (Beta) and LLOD, and a correlation between n and LLOD.

10. The gas sensing method of claim 1, wherein in the operation v), the standard correlation of the two or more kinetic parameters or static parameters is data obtained by repeating the operations of i) to iv) a plurality of times for a target gas to be measured and calculating the correlation of the two or more kinetic or static parameters of the target gas.

11. The gas sensing method of claim 1, wherein the operation v) comprises visualizing the correlation of the two or more kinetic parameters or static parameters as a scatter plot and comparing the visualized correlation with a standard scatter plot visualizing the standard correlation of the two or more kinetic parameters or static parameters.
